(19) United States
(12) Patent Application Publication
(10) Pub. No.: US 2026/0076374 A1
(43) Pub. Date: Mar. 19, 2026

(54) METHODS AND COMPOSITIONS FOR REDUCING DAMAGE TO A PLANT OR PLANT PART

(71) Applicant: NewLeaf Symbiotics, Inc., St. Louis, MO (US)

(72) Inventors: David Flack, Upper Arlington, OH (US); Desmond R. Jimenez, Woodland, CA (US); Anne ZIMMERMAN PHILLIPS, St. Louis, MO (US)

(21) Appl. No.: 18/996,009

(22) PCT Filed: Jul. 19, 2023
(86) PCT No.: PCT/US2023/070468

§ 371 Date: Jan. 17, 2025

Related U.S. Application Data
(60) Provisional application No. 63/368,783, filed on Jul. 19, 2022.

(51) Int. Cl. A01N 63/20; A01P 7/04; C12N 1/20; C12R 1/01

(52) U.S. Cl. CPC A01N 63/20; A01P 7/04; C12N 1/205; C12R 2001/01

(57) ABSTRACT

The present disclosure provides compositions comprising Methylobacterium strains and methods related thereto that reduce plant damage due to piercing-sucking insects and/or pathogens transmitted by such insects.

### CROSS-REFERENCE TO RELATED APPLICATIONS

This patent application claims the benefit of U.S. Provisional Patent Application No. 63/368,783, filed Jul. 19, 2022: the entire disclosure of which are incorporated herein by reference.

### BACKGROUND

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase, that incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Most methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nidulans is a nitrogen-fixing Methylobacterium that is not a PPFM (Sy et al., 2001). Methylobacterium are ubiquitous in nature, being found in soil, dust, fresh water, sediments, and leaf surfaces, as well as in industrial and clinical environments (Green, 2006).

### SUMMARY

Provided herein are Methylobacterium strains NLS0017, deposited as NRRL B-50931, and or NLS0109, deposited as NRRL B-67340, and their use in plant treatment methods and compositions to reduce damage to a plant caused by an insect and/or a disease caused by a pathogen transmitted by an insect, wherein said insects are piercing-sucking insects. Also provided are compositions comprising such Methylobacterium strains or combinations of strains, methods of using the compositions to treat plants, plant parts, or soil where plants are grown to reduce piercing-sucking insect damage to plants, plant parts, and plants derived therefrom, and/or to reduce damage to plants caused by plant pathogens vectored by such insects.

Also provided herein are methods for reducing virus damage to a plant wherein said virus is transmitted by a piercing-sucking insect. In such methods, a composition comprising Methylobacterium strain NLS0017 and/or NLS0109 is applied to a plant, plant part, such as a plant seed, or to soil where a plant seed has been or will be planted, in a location where populations of piercing-sucking insects exist or are expected to exist during the growing season of the plant. In some embodiments, the plant is a tomato or peanut plant. In some embodiments, the piercing-sucking insect is a leafhopper or a thrips. In some embodiments the leafhopper is a beet leafhopper. In some embodiments, the thrips is a western flower thrips or a tobacco thrips. In some embodiments, the transmitted virus is a tospovirus or a geminivirus. In some embodiments, the tospovirus is tomato spotted wilt virus (“TSWV”). In some embodiments, the geminivirus is beet curly top virus.

Also provided is a virus disease management process for use in growing a crop in a region where insects capable of transmitting a virus that infects said crop is present. The process comprises applying a composition comprising Methylobacterium NLS0017 and/or NLS0109 to a plant, a seed, or soil where said seed has been or will be planted: applying an insecticide active against said insect to said plant, seed or soil where said seed has been or will be planted, and growing said plant or seed in the presence of said insect, thereby reducing damage to said plant from said virus as compared to a plant not treated with said composition or grown in soil not treated with said composition. In some embodiments, the amount of said insecticide is reduced in comparison to the recommended use for reducing populations of said insects in a crop grown in the region. In some embodiments, the plant is a peanut plant, and Methylobacterium NLS0017 and/or NLS0109 is applied to a peanut seed, or to soil where said peanut seed will be grown. In some embodiments, the plant is a tomato plant, and Methylobacterium NLS0017 and/or NLS0109 is applied to a seedling as a drench prior to transplanting to a field.

In any of the above methods, the composition can comprise one or more additional Methylobacterium strains. In some embodiments, an additional Methylobacterium is selected from the group consisting of NLS0020 deposited as NRRL B-50930, NLS0064 deposited as NRRL B-50938, and NLS0038 deposited as NRRL B-50942 is present in said composition. In some embodiments, one or more additional Methylobacterium strains are selected from the deposited strains listed in Table 1 herein.

In certain embodiments of the methods, the composition comprises NLS0017 and/or NLS0109 at a titer of at least 5×10⁶ colony-forming units (CFU) per gram of a dried composition or at least 5×10⁶ CFU per/ml of a liquid composition. In some embodiments NLS0017 and NLS0109 are each present in a composition at about 5×10⁸, 1×10⁹, or 1×10¹⁰ to about 5×10¹³ CFU per gram in a solid composition, or at a titer of about 1×10⁶ CFU/mL to about 1×10⁹ CFU/mL for a liquid composition. In other embodiments comprising 2 or more Methylobacterium strains, the total concentration of Methylobacterium strains in the compositions is at least 5×10⁶ CFU per gram of a dried composition or at least 5×10⁶ CFU per/ml of a liquid composition. In some embodiments, two or more Methylobacterium strains are provided in the compositions in different amounts. In some embodiments, compositions comprise primarily NLS0017. In some embodiments, compositions comprise primarily NLS0109. In some embodiments, compositions comprise a 75:25 mixture of NLS0017 and NLS0109. In some embodiments, compositions comprise a 25:75 mixture of NLS0017 and NLS0109. In some embodiments, NLS0017 and NLS0109 are provided as 90:10, 80:20, 70:30, 60:40, 40:60, 30:70, 20:80, or 10:90 mixtures of the strains.

In certain embodiments, the applied composition coats or partially coats the plant or plant part, where the plant part can be, for example, a plant seed. In certain embodiments, a composition is applied to soil by broadcasting the composition, by drenching the soil with the composition, and/or by depositing the composition in a furrow where seed is or will be planted. In certain embodiments, a composition comprising NLS0017 and/or NLS0109 is applied to a plant seedling as a drench prior to or concurrent with planting said seedling in a field. In certain embodiments of the methods, the composition is applied to foliage of the plant. In certain embodiments, the composition further comprises an insecticide that is active against piercing-sucking insects.

### BRIEF DESCRIPTION OF THE DRAWINGS

The accompanying drawings, which are incorporated in and form a part of the specification, illustrate certain embodiments of the present disclosure. In the drawings:

FIG. 1 compares “TREATED”-tomato plants grown from seedlings treated as a drench prior to planting with a composition comprising NLS0017 and NLS0109, and untreated control tomato plants (UTC) grown from untreated transplanted seedlings.

### DESCRIPTION

### Definitions

As used herein, the phrase “agriculturally acceptable adjuvant” refers to a substance that enhances the performance of an active agent in a composition for treatment of plants and/or plant parts. In certain compositions, an active agent can comprise a mono-culture or co-culture of Methylobacterium.

As used herein, the phrase “agriculturally acceptable excipient” refers to an essentially inert substance that can be used as a diluent and/or carrier for an active agent in a composition for treatment of plants and/or plant parts.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

As used herein, a “leafy green plant” refers to a vegetable crop with edible leaves and includes, without limitation, spinach, kale, lettuce (including but not limited to romaine, butterhead, iceberg, and loose leaf lettuces), collard greens, cabbage, beet greens, watercress, swiss chard, arugula, escarole, endive, bok choy, and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, beet, spinach, melon, cucumber, squash, basil, celery, cilantro, radish, radicchio, chicory, dill, rosemary, French tarragon, basil, carrot, fennel, beans, peas, chickpeas, and lentils. Leafy green plants also refer to mixes of assorted leafy green plants, such as mesclun or other mixed salad greens or mixed microgreens. “Leafy green plants” as used herein also encompasses other brassica or cruciferous field greens not specifically mentioned herein by name.

As used herein, a “fruit” or “fruit bearing plant” is a fleshy fruit bearing plant, including but not limited to, melon (including watermelon and cantaloupe), berry (including strawberry, blueberry, blackberry, and raspberry), grape, kiwi, mango, papaya, pineapple, banana, pepper, tomato, squash, and cucumber plants.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extor quens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense); Methylobacterium mesophilicum; Methylobacterium thiocyanatum (Methylorubrum thiocyanatum); Methylobacterium nodulans; Methylobacterium zatmanii (Methylorubrum zatmanii); or Methylobacterium organophilum.

As used herein, the phrase “essentially free of contaminating microorganisms” refers to a culture, fermentation broth, fermentation product, or composition where at least about 95% of the microorganisms present by amount or type in the culture, fermentation broth, fermentation product, or composition are the desired Methylobacterium or other desired microorganisms of pre-determined identity.

As used herein, the phrase “mono-culture of Methylobacterium” refers to a Methylobacterium culture consisting of a single strain of Methylobacterium.

As used herein, a “pesticide” refers to an agent that is insecticidal, fungicidal, nematocidal, bacteriocidal, or any combination thereof.

As used herein, the phrase “bacteriostatic agent” refers to agents that inhibit growth of bacteria but do not kill the bacteria.

As used herein, the phrase “pesticide does not substantially inhibit growth of said Methylobacterium” refers to any pesticide that when provided in a composition comprising a fermentation product comprising a solid substance wherein a mono-culture or co-culture of Methylobacterium is adhered thereto, results in no more than a 50% inhibition of Methylobacterium growth when the composition is applied to a plant or plant part in comparison to a composition lacking the pesticide. In certain embodiments, the pesticide results in no more than a 40%, 20%, 10%, 5%, or 1% inhibition of Methylobacterium growth when the composition is applied to a plant or plant part in comparison to a composition lacking the pesticide.

As used herein a piercing-sucking insect is one that feeds on plants by piercing cells or vascular tissues with specialized mouthparts and sucking the contents. Many piercing-sucking insects have a stylus-like mouthpart (also referred to as a beak or proboscis) to pierce the plant and suck out liquids. Other insects considered piercing-sucking insects herein, such as thrips, suck up plant fluids after rasping or scraping with their mouthparts to pierce plant.

As used herein “fertilizer” can be a single nutrient nitrogen fertilizer, such as urea, ammonia, or ammonia solutions (including ammonium nitrate, ammonium sulfate, calcium ammonium nitrate, and urea ammonium nitrate). In certain embodiments, the fertilizer can be a single nutrient phosphate fertilizer, such as a superphosphate or triple superphosphate or mixtures thereof, including double superphosphate. In certain embodiments, the fertilizer can be a single nutrient potassium-based fertilizer, such as muriate of potash. In certain embodiments, the compositions comprise multinutrient fertilizers including binary fertilizers (NP, NK, PK), including, for example monoammonium phosphate, diammonium phosphate, potassium nitrate, and potassium chloride. In further embodiments, three-component fertilizers (NPK) providing nitrogen, phosphorus, and potassium are present in the aqueous compositions. In still further embodiments, the fertilizer comprises micronutrients, which may be chelated or non-chelated. In some embodiments, combinations of various fertilizers can be present in the aqueous solution, including combinations of nitrogen, phosphorus, and/or micronutrient fertilizers. Nutrient solutions provided in hydroponic plant growth systems are also considered “fertilizers” in methods and compositions described herein.

As used herein, the term “strain” shall include all isolates of such strain.

As used herein, “variant” when used in the context of a Methylobacterium isolate, refers to any isolate that has chromosomal genomic DNA with at least 99%, 99.9%, 99.8%, 99.7%, 99.6%, or 99.5% sequence identity to chromosomal genomic DNA of a reference Methylobacterium isolate, such as, for example, a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinidis et al. (2005)) using sequence analysis tools, such as BLAST, as taught by Altschul et al. (1990) or clustalw (www.ebi.ac.uk/Tools/msa/clustalw2/). Variants can be identified, for example, by the presence of a 16S sequence of a reference strain, where the variant also demonstrates a plant production enhancement trait of the reference strain.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Methylobacterium Strains Providing Protection to Insect Damage and Damage Due to Insect Transmitted Disease

Methylobacterium NLS0017 and/or NLS0109 are demonstrated herein to provide protection to plants against damage from insect feeding and/or diseases caused by plant pathogens transmitted by said insects, where the insects are piercing-sucking insect that feed on plants by piercing cells or vascular tissues with specialized mouthparts and sucking the contents. Effects of NLS0017 and/or NLS0109 can be demonstrated by increased yield of plants treated with NLS0017 and/or NLS0109 and/or by decreased symptoms associated with plant damage due to a pathogen transmitted, or vectored, by said insect. In some embodiments, application of NLS0109 with NLS0017 increases the protective effect of either strain alone on treated plants. In some embodiments, an additional Methylobacterium strain can be applied, for example a strain selected from the group consisting of NLS0020, NLS0064, NLS0038 and other strains provided in Table I herein. In some embodiments, NLS0109 alone is used to treated plants and provide protection against piercing-sucking insects and or pathogens transmitted by such insects. In some embodiments, compositions for plant treatment comprise additional components, including for example, insecticides active against said piercing-sucking insects.

Effects of Methylobacterium NLS0017 and/or NLS0109 on piercing-sucking insects and pathogens vectored by such insects may result from various effects or combinations of effects of the Methylobacterium on the treated plant. Feeding from piercing-sucking insects can cause spotting or stippling of foliage, leaf curling, and stunted or misshapen fruits, in addition to effects caused by vectored pathogens. Some plant growth promoting rhizobacteria (PGPR) are capable of eliciting broad-spectrum induced systemic resistance (ISR). Some PGPR strains elicit responses similar to pathogen-induced systemic acquired resistance (SAR). PGPR treatment may also interfere with viral disease progress in the plant. Some PGPRs affect viral disease progression via salicylic acid- and jasmonic acid-dependent pathways. Some trigger (ISR), secrete endonucleases and regulate phytohormones. Some PGPRs increase the content of cytokinin, decrease levels of indole acetic acid (IAA) or increase abscisic acid (ABA). These interactions can trigger protection against plant virus disease progression. PGPRs have also been reported to alter plant volatile compounds emissions and thus reduce the attraction of insects to the plant. Thus, although the exact mechanism by which NLS0017 and/or NLS0109 provide protection against insects and insect vectored pathogens is not known, the examples provided herein demonstrate such effects are substantial and greater than seed with other PGPR tested herein, including strains of Rhizobium.

Effects of Methylobacterium on treated plants and/or plants grown from treated seed, may be evaluated in a number of ways, including by measurements of plant damage, detection of insect probing events, for example by staining (Backus (1988) Journal of Economic Entomology 81:1819-1823), quantification of viral presence in plant tissues, for example by sequencing, immunological and/or genetic hybridization or amplification techniques. Amplification techniques include, but are not limited to PCT, RT-PCR, and loop mediated isothermal amplification (LAMP). Hyperspectral imaging (see, for example Lowe et el. (2017) Plant Methods 13:80; Manganiello et al. (2021) Frontiers in Plant Science 12:630059), and choice and non-choice assays (Weintraub et al. (2008) Journal of Economic Entomology 101:1337-1340) may also be used to evaluate the effects of Methylobacterium treatments on treated plants.

In certain embodiments of the methods provided herein, the composition further comprises an insecticide active against piercing-sucking insects. In some embodiments, the insecticide is a systemic insecticide, such as acephate, phorate, imidacloprid or disulfoton. Other insecticides that may be used include pyrethrins, endosulfan, phoxim, thiamethoxam, malathion and bifenthrin. Insecticides that kill insects by contact and ingestion, such as butylene fipronil, chlorpyrifos, chlorfenapyr, and benfuracarb may also be used, for example to control thrips in a greenhouse environment.

In certain embodiments of the methods provided herein, Methylobacterium NLS0017 and/or NLS0109 provide protection against piercing-sucking insects that transmit viral pathogens. In certain embodiments of the methods provided herein provide protection against insects, including insects selected from the group consisting of leafhoppers, thrips and aphids. In certain embodiments of the methods provided herein provide protection against a transmitted virus, including a virus selected from the group consisting of a tospovirus and a geminivirus. In some embodiments, a treated plant is a peanut plant, a piercing-sucking insect is a western flower thrips or a tobacco thrips, and a virus is tomato spotted wilt virus. In some embodiments, a treated plant is a tomato plant, a piercing-sucking insect is a beet leafhopper, and a virus is beet curly top virus.

Treatment with NLS0017 and/or NLS0109 as described herein will also find use with a broad range of plants that are affected by piercing-sucking insects and/or pathogens transmitted by such insects including viral and bacterial pathogens. Aphids, leafhoppers, stink bugs, tarnished plant bugs, squash bugs, and thrips. Protection can also be provided for plant pests that are closely related insects, for example spider mites, that also attack plants and suck liquids from the leaves.

Various Methylobacterium strains provided herein that reduce insect damage and/or insect transmitted disease damage to a crop, or find use in combination with such Methylobacterium strains are disclosed in Table 1.

TABLE 1
Methylobacterium Strains

| | | USDA ARS |
|---|---|---|
| Deposit Identifier | NLS ID | NRRL No.¹ |
| Methylobacterium sp. #1 | NLS0046 | NRRL B-50929 |
| Methylobacterium sp. #2 | NLS0020 | NRRL B-50930 |
| Methylobacterium sp. #3 | NLS0017 | NRRL B-50931 |
| Methylobacterium sp. #4 | NLS0042 | NRRL B-50932 |
| Methylobacterium sp. #5 | NLS0089 | NRRL B-50933 |
| Methylobacterium sp. #6 | NLS0068 | NRRL B-50934 |
| Methylobacterium sp. #7 | NLS0065 | NRRL B-50935 |
| Methylobacterium sp. #8 | NLS0069 | NRRL B-50936 |
| Methylobacterium sp. #9 | NLS0062 | NRRL B-50937 |
| Methylobacterium sp. #10 | NLS0064 | NRRL B-50938 |
| Methylobacterium sp. #11 | NLS0021 | NRRL B-50939 |
| Methylobacterium sp. #12 | NLS0066 | NRRL B-50940 |
| Methylobacterium sp. #13 | NLS0037 | NRRL B-50941 |
| Methylobacterium sp. #14 | NLS0038 | NRRL B-50942 |
| Methylobacterium sp. #15 | NLS0044 | NRRL B-67339 |
| Methylobacterium sp. #16 | NLS0109 | NRRL B-67340 |
| Methylobacterium sp. #17 | NLS0934 | NRRL B-67341 |
| Methylobacterium sp. #18 | NLS0648 | NRRL B-67741 |
| Methylobacterium sp. #19 | NLS0662 | NRRL B-67742 |
| Methylobacterium sp. #20 | NLS0807 | NRRL B-67743 |
| Methylobacterium sp. #21 | NLS0476 | NRRL B-67809 |
| Methylobacterium sp. #26 | NLS0610 | NRRL B-67892 |
| Methylobacterium sp. #22 | NLS0497 | NRRL B-67925 |
| Methylobacterium sp. #23 | NLS0693 | NRRL B-67926 |
| Methylobacterium sp. #25 | NLS1181 | NRRL B-67927 |
| Methylobacterium sp. #24 | NLS1179 | NRRL B-67929 |
| Methylobacterium sp. #28 | NLS0433 | NRRL B-68032 |
| Methylobacterium sp. #29 | NLS1026 | NRRL B-68033 |
| Methylobacterium sp. #30 | NLS1084 | NRRL B-68034 |
| Methylobacterium sp #32 | NLS0687 | NRRL B-68065 |
| Methylobacterium sp #33 | NLS0722 | NRRL B-68066 |
| Methylobacterium sp #34 | NLS0834 | NRRL B-68067 |
| Methylobacterium sp #35 | NLS0940 | NRRL B-68068 |
| Methylobacterium sp #36 | NLS0947 | NRRL B-68069 |
| Methylobacterium sp. #37 | NLS0737 | NRRL-B-68074 |
| Methylobacterium sp. #38 | NLS0770 | NRRL-B-68075 |
| Methylobacterium sp. #39 | NLS5278 | NRRL-B-68186 |
| Methylobacterium sp. #40 | NLS5334 | NRRL-B-68187 |
| Methylobacterium sp. #41 | NLS5480 | NRRL-B-68188 |
| Methylobacterium sp. #42 | NLS5549 | NRRL-B-68189 |
| Methylobacterium sp #43 | NLS0665 | NRRL-B-68194 |
| Methylobacterium sp #44 | NLS0729 | NRRL-B-68195 |
| Methylobacterium sp #45 | NLS0672 | NRRL-B-68196 |
| Methylobacterium sp #46 | NLS0754 | NRRL-B-68197 |
| Methylobacterium sp #47 | NLS0591 | NRRL-B-68215 |
| Methylobacterium sp #48 | NLS0439 | NRRL-B-68216 |
| Methylobacterium sp #49 | NLS1310 | NRRL-B-68217 |
| Methylobacterium sp #50 | NLS1312 | NRRL-B-68218 |
| Methylobacterium sp #51 | NLS0049 | NRRL-B-68236 |
| Methylobacterium sp #52 | NLS0612 | NRRL-B-68237 |
| Methylobacterium sp #53 | NLS0706 | NRRL-B-68238 |
| Methylobacterium sp #54 | NLS0725 | NRRL-B-68239 |
| Methylorubrum sp. #63 | NLS7725 | NRRL B-68260 |

¹Deposit number for strain deposited with the AGRICULTURAL RESEARCH SERVICE

CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR § 1.808 (b), all restrictions imposed by the depositor on the availability to the public of the deposited material will be irrevocably removed upon the granting of any patent from this patent application.

Variants of a Methylobacterium isolate listed in Table I may also find use including isolates obtained therefrom by genetic transformation, mutagenesis, and/or insertion of a heterologous sequence. In some embodiments, such variants are identified by the presence of chromosomal genomic DNA with at least 99%, 99.9%, 99.8%, 99.7%, 99.6%, or 99.5% sequence identity to chromosomal genomic DNA of the strain from which it was derived.

Methods for reducing damage from piercing-sucking insects and/or pathogens vectored by such insects comprise applying any of the aforementioned compositions to a plant or a plant part in an amount that provides for inhibition of insect or vectored pathogen damage in the plant, plant part, or a plant obtained therefrom relative to infection of a control plant, plant part, or plant obtained therefrom that had not received an application of the composition. In certain embodiments, application of the composition provides for at least about 5%, at least about 10%, at least about 20%, at least about 30%, at least about 40%, at least about 50%, at least about 75%, at least about 85%, or at least about 95% reduction of damage in the plant, plant part, or a plant derived therefrom relative to the control plant, plant part, or plant obtained therefrom. In certain embodiments, a treated plant is a plant seedling, or a treated plant part is selected from the group consisting of a leaf, a stem, a flower, a root, a tuber, and a seed.

Treatments or applications to plants described herein can include, but are not limited to, spraying, coating, partially coating, immersing, and/or imbibing the seed, plant, or plant parts with the Methylobacterium strains and compositions comprising the same provided herein. In certain embodiments, soil, a seed, a leaf, a stem, a root, a tuber, or a shoot can be sprayed, immersed, and/or imbibed with a liquid, semi-liquid, emulsion, or slurry of a composition provided herein. In other embodiments, the Methylobacterium composition is applied to soil by broadcasting the composition, by drenching the soil with the composition, and/or by depositing the composition in a furrow where said seed is or will be planted.

In certain embodiments where plant seeds are treated with Methylobacterium compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soy bean protein powders, or a combination of protein powders and a lipid, for example lecithin or a vegetable oil. Lubricants can be applied to seeds simultaneously with application of Methylobacterium, or may be mixed with Methylobacterium prior to application of the compositions to the seeds.

In certain embodiments, treated plants are cultivated in a hydroponic system. In some embodiments, plant seeds are treated and plants are grown from the treated seeds continuously in the same cultivation system. In some embodiments, plant seeds are treated and cultivated in a hydroponic nursery to produce seedlings. The seedlings can be drenched and planted in a field following treatment, or can be treated and transferred to a different hydroponic system, for example for commercial production of leafy greens.

In certain embodiments, the composition used to treat the seed, plant or plant part can contain a Methylobacterium strain and an agriculturally acceptable excipient. Agriculturally acceptable excipients include, but are not limited to, woodflours, clays, activated carbon, diatomaceous earth, fine-grain inorganic solids, calcium carbonate, and the like. Clays and inorganic solids that can be used include, but are not limited to, calcium bentonite, kaolin, china clay, talc, perlite, mica, vermiculite, silicas, quartz powder, montmorillonite, and mixtures thereof. Agriculturally acceptable excipients also include various lubricants such as talc, graphite, polyethylene wax based powders (such as Fluency Agent), protein powders, for example soybean protein powders, or a combination of protein powders and a lipid, for example lecithin or a vegetable oil.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soy bean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene, and syrups or mixtures thereof. Other useful agriculturally acceptable adjuvants that can promote coating include, but are not limited to, polymers and copolymers of vinyl acetate, polyvinylpyrrolidone-vinyl acetate copolymer, and water-soluble waxes. Further, agriculturally acceptable adjuvants also include various lubricants (wich can provide for smooth flow and separation (singulation) of seeds) such as talc, graphite, polyethylene wax based powders (such as Fluency Agent), protein powders, for example soybean protein powders, or a combination of protein powders and a lipid, for example lecithin or a vegetable oil. Various surfactants, dispersants, anticaking-agents, foam-control agents, and dyes disclosed herein and in U.S. Pat. No. 8,181,388 can be adapted for use with compositions comprising a suitable Methylobacterium strain. In certain embodiments, the seed and/or seedling is exposed to the composition by providing the Methylobacterium strain in soil in which the plant or a plant arising from the seed are grown, or other plant growth media in which the plant or a plant arising from the seed are grown. Examples of methods where the Methylobacterium strain is provided in the soil include in furrow applications, soil drenches, and the like.

Non-limiting examples of treatments of plant seeds, seedling, or other plant parts with a Methylobacterium include treatments of vegetable crops with edible leaves including, without limitation, spinach, kale, lettuce (including but not limited to romaine, butterhead, iceberg and loose leaf lettuces), and field greens, including brassica greens. Specific greens that can be treated with Methylobacterium provided herein include collard greens, cabbage, beet greens, watercress, swiss chard, arugula, escarole, endive, bok choy, and turnip greens. Other leafy green plants that are grown for production and harvest of microgreens and/or herbs, can also be treated in the methods described herein, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils. Treatment of plants grown for harvest of fleshy fruits are also provided herein. Such plants include, for example, melon (including watermelon and cantaloupe), berry (including strawberry, blueberry, blackberry, and raspberry), grape, kiwi, mango, papaya, pineapple, banana, pepper, tomato, squash, and cucumber plants. Other plants that can be treated include, for example field crops, ornamentals, turf grasses, and trees grown in commercial production, such as conifer trees. Without limitation, such additional plant species include corn, soybean, cruciferous or Brassica sp. vegetables (e.g., B. napus, B. rapa, B. juncea), alfalfa, rice, rye, wheat, barley, oats, sorghum, millet (e.g., pearl millet (Pennisetum glaucum), proso millet (Panicum miliaceum), foxtail millet (Setaria italica), and finger millet (Eleusine coracana)), sunflower, safflower, tobacco, potato, peanuts, cotton, species in the genus Cannabis (including, but not limited to, Cannabis sativa and industrial hemp varieties), sweet potato (Ipomoea batatus), cassava, coffee, coconut, ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar), and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass).

In certain embodiments, a manufactured combination composition comprising two or more Methylobacterium strains can be used to treat a seed, plant or plant part in any of the methods provided herein. Such manufactured combination compositions can be made by methods that include harvesting monocultures of each Methylobacterium strain and mixing the harvested monocultures to obtain the manufactured combination composition of Methylobacterium. In certain embodiments, the manufactured combination composition of Methylobacterium can comprise Methylobacterium isolated from different plant species or from different cultivars or varieties of a given plant.

In certain embodiments, an effective amount of the Methylobacterium strain or strains used in treatment of plants, seeds, or plant parts is a composition having a Methylobacterium titer of at least about 1×10⁶ colony-forming units per milliliter, at least about 5×10⁶ colony-forming units per milliliter, at least about 1×10⁷ colony-forming units per milliliter, at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an effective amount of the Methylobacterium strain or strains is a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5×10⁸ colony-forming units per milliliter to at least about 6×10¹⁰ colony-forming units per milliliter of a liquid or an emulsion. In certain embodiments, an effective amount of the Methylobacterium strain or strains is a composition with the Methylobacterium at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of the composition. In certain embodiments, an effective amount of a composition provided herein can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of Methylobacterium strain or strains is adhered thereto. In certain embodiments, an effective amount of a composition provided herein to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains adhered to a solid substance is provided therein or grown therein. In certain embodiments, an effective amount of a composition provided herein can be a composition with a Methylobacterium titer of at least about 1×10⁶ colony-forming units per mL, at least about 5×10⁶ colony-forming units per mL, at least about 1×10⁷ colony-forming units per mL, or at least about 5×10⁸ colony-forming units per mL to at least about 6×10¹⁰ colony-forming units of Methylobacterium per mL in a composition comprising an emulsion wherein a mono-culture or co-culture of a Methylobacterium strain or strains is provided therein or grown therein. In certain embodiments, any of the aforementioned compositions comprising a mono-culture or co-culture of a Methylobacterium strain or strains can further comprise a mono- or co-culture of Rhizobium and/or Bradyrhizobium.

In certain embodiments, an effective amount of a Methylobacterium strain or strains provided in a treatment of a seed or plant part is at least about 10³, 10⁴, 10⁵, or 10⁶ CFU per seed or treated plant part. In certain embodiments, an effective amount of Methylobacterium provided in a treatment of a seed or plant part is at least about 10³, 10⁴, 10⁵, or 10⁶ CFU to about 10⁷, 10⁸, 10⁹, or 10¹⁰ CFU per seed or treated plant part. In certain embodiments, the effective amount of Methylobacterium provided in a treatment of a seed or plant part is an amount where the CFU per seed or treated plant part will exceed the number of CFU of any resident naturally occurring Methylobacterium strain by at least 5-10-, 100-, or 1000-fold. In certain embodiments, the effective amount of Methylobacterium provided in a treatment of a seed or plant part is an amount where the CFU per seed or treated plant part will exceed the number of CFU of any resident naturally occurring Methylobacterium by at least 2-, 3-, 5-, 8-, 10-, 20-, 50-, 100-, or 1000-fold. In certain embodiments where the treated plant is cultivated in a hydroponic system, populations of naturally occurring Methylobacterium or other soil microbes will be minimal.

In some embodiments, the composition or method disclosed herein may comprise one or more additional components. In some embodiments a second component can be an additional active ingredient, for example, a pesticide, a non-biological plant or microbial stimulant, or a second biological, including other beneficial bacteria and beneficial fungi. The pesticide may be, for example, an insecticide, a fungicide, an herbicide, or a nematicide. The second biological can be a biocontrol agent. Additional ingredients can also include fertilizers.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT): 2-Bromo-2-nitro-propane-1,3-diol (Bronopol), 5-bromo-5-nitro-1,3-dioxane (Bronidox), Tris(hydroxymethyl) nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins. Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a Methylobacterium strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a plant or microbial biostimulant. In some embodiments, an additional active ingredient may be a second biological. The second biological could be a biocontrol agent, other beneficial microorganisms, microbial extracts, plant extracts (botanicals), and other natural products, including for example, protein hydrolysates and other nitrogen containing compounds, plant growth activators or stimulants or a plant defense agent. Examples of useful additional components include yeast or seaweed or extracts and powders thereof, humic and fulvic acids, amino acids, peptides, chitosan and other biopolymers, plant hormones, such as auxins and cytokinins or derivatives thereof, trace elements and nucleic acids. In some embodiments, compositions provided here contain combinations of any of these or related compounds. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In further embodiments the second biological can be plant growth activators or plant defense agents including, but not limited to harpin, Reynoutria sachalinensis, jasmonate, lipochitooligosaccharides, and isoflavones.

Agriculturally acceptable adjuvants used in the compositions that comprise Methylobacterium include, but are not limited to, components that enhance product efficacy and/or products that enhance ease of product application. Adjuvants that enhance product efficacy can include various wetters/spreaders that promote adhesion to and spreading of the composition on plant parts, stickers that promote adhesion to the plant part, penetrants that can promote contact of the active agent with interior tissues, extenders that increase the half-life of the active agent by inhibiting environmental degradation, and humectants that increase the density or drying time of sprayed compositions. Wetters/spreaders used in the compositions can include, but are not limited to, non-ionic surfactants, anionic surfactants, cationic surfactants, amphoteric surfactants, organo-silicate surfactants, and/or acidified surfactants. Stickers used in the compositions can include, but are not limited to, latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve ease of product application include, but are not limited to, acidifying/buffering agents, anti-foaming/de-foaming agents, compatibility agents, drift-reducing agents, dyes, and water conditioners. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

### Deposit Information

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339 was deposited with NRRL on Nov. 18, 2016. Methylobacterium sp. NRRL B-67340 was deposited with NRRL on Nov. 18, 2016. Methylobacterium sp. NRRL B-67341 was deposited with NRRL on Nov. 18, 2016. Methylobacterium sp. NRRL B-67741 was deposited with NRRL on Dec. 20, 2018. Methylobacterium sp. NRRL B-67742 was deposited with NRRL on Dec. 20, 2018. Methylobacterium sp. NRRL B-67743 was deposited with NRRL on Dec. 20, 2018. Methylobacterium sp. NRRL B-67809 was deposited with NRRL on Jun. 28, 2019. Methylobacterium sp. NRRL B-67892 was deposited with NRRL on Nov. 26, 2019. Methylobacterium sp. NRRL B-67925, NRRL B-67926, and NRRL B-67927 were deposited with NRRL on Feb. 21, 2020. Methylobacterium sp. NRRL B-67929 was deposited with NRRL on Mar. 3, 2020. Methylobacterium sp. NRRL B-68032, NRRL B-68033 and NRRL B-68034 were deposited with NRRL on May 20, 2021. Methylobacterium sp. NRRL B-68065, NRRL B-68066, NRRL B-68067, NRRL B-68068, and NRRL B-68069 were deposited with NRRL on Sep. 9, 2021. Methylobacterium sp. NRRL B-68074 and NRRL B-68075 were deposited with NRRL on Oct. 6, 2021. Methylobacterium sp. NRRL B-68186, NRRL B-68187, NRRL B-68188 and NRRL B-68189 were deposited with NRRL on Aug. 3, 2022. Methylobacterium sp. NRRL B-68194, NRRL B-68195, NRRL B-68196, and NRRL B-68197 were deposited with NRRL on Aug. 30, 2022. Methylobacterium sp. NRRL B-68215, NRRL B-68216, NRRL B-68217, and NRRL B-68218 were deposited with NRRL on Nov. 2, 2022. Methylobacterium sp. NRRL B-68236, NRRL B-68237, NRRL B-68238, and NRRL B-68239 were deposited with NRRL on Nov. 23, 2022. Methylorubrum sp. NRRL B-68260 was deposited with NRRL on Mar. 9, 2023.

Subject to 37 CFR § 1.808 (b), all restrictions imposed by the depositor on the availability to the public of the deposited material will be irrevocably removed upon the granting of any patent from this patent application.

### EXAMPLES

The following examples are included to demonstrate certain embodiments. It will be appreciated by those of skill in the art that the techniques disclosed in the following examples represent techniques determined by the Applicants to function well in the practice of the disclosure. However, those of skill in the art should, in light of the instant disclosure, appreciate that many changes can be made in the specific embodiments that are disclosed, while still obtaining like or similar results, without departing from the scope of the invention.

### Example 1. Methylobacterium Application to Peanut

A peanut trial was established at Tallahassee, FL at two locations to evaluate effects of Methylobacterium strains applied as in-furrow treatments at seeding on yield and tomato spotted wilt virus (TSWV) severity in peanut. The panhandle area of Florida has historically experienced high levels of tomato spotted wilt virus infestation and attendant reduced yields and damage to peanut crops.

The field trial was conducted as a randomized complete block consisting of four 30-inch rows that were each 40 feet long. There were four replications of each of 12 treatments at each location, including three treatments with single Methylobacterium strains, NLS0017 (NRRL B-50931), NLS0020 (NRRL B-50930) and NLS0064 (NRRL B-50938); two combinations with two different strains of Methylobacterium, NLS0017+NLS0020, and NLS0017+NLS0038 (NRRL B-50942); and three treatments with combinations of Methylobacterium strains and a strain of Rhizobia. Controls included untreated, Rhizobia only treatment, and Rhizobia+2 different peanut commercial Rhizobia seed treatments. Data were collected from the two center rows. Soil series were recorded and fertility was measured from 5 core samples at a depth of 7 inches. Soil surface and two-inch depth temperature and moisture data were collected at planting. Historic weather (2 months before planting) and daily minimum/maximum temperatures were recorded. Rainfall and irrigation were recorded. Stand count and vigor (indexed scale 1-9) were collected seven and twenty-one days after planting. Flowering date and pegging were recorded as the date after planting when 50% were pegged or had exposed flowers. Lodging was recorded at harvest and yield was estimated as pounds per acre. Test weight and seed moisture data were collected. TSWV severity was assessed at each location and scored on a scale of 1 to 10 disease intensity based on a visual rating of typical symptoms.

Results are shown in Tables 2 and 3 below.

TABLE 2
Yield Measurements

| | | Yield | Yield | |
|---|---|---|---|---|
| | | (LBS/AC) | (LBS/AC) | |
| | trt | Location 1 | Location 2 | average |
| UNT | 1 | 3702.6 | 2262.5 | 2982.5 |
| Rhiz | 2 | 4229 | 2634.6 | 3431.8 |
| Vault | 3 | 4404.4 | 3084.7 | 3744.5 |
| Optimize | 4 | 4416.5 | 2838.6 | 3627.5 |
| NLS0017 (NRRL B-50931) | 5 | 4967.1 | 3138.7 | 4052.9 |
| NLS0020 (NRRL B-50930) | 6 | 4241.1 | 3354.7 | 3797.9 |
| NLS0064 (NRRL B-50938) | 7 | 4192.7 | 3396.7 | 3794.7 |
| NLS0017/NLS0020 | 8 | 5263.5 | 3474.7 | 4369.1 |
| NLS0017/NLS0038 | 9 | 4815.8 | 2844.6 | 3830.2 |
| NLS0017 + Rhiz | 10 | 5523.7 | 3606.8 | 4565.2 |
| NLS0020 + Rhiz | 11 | 4706.9 | 3828.8 | 4267.9 |
| NLS0064 + Rhiz | 12 | 4967.1 | 3540.7 | 4253.9 |
| Avg × Loc | | 4619.2 | 3167.2 | 3893.2 |

TABLE 3
TSWV Severity Assessment

| | | TSWV | TSWV | |
|---|---|---|---|---|
| | trt | Location 1 | Location 2 | average |
| UNT | 1 | 9.1 | 3.3 | 6.2 |
| Rhiz | 2 | 7.1 | 1.7 | 4.4 |
| Vault | 3 | 4.2 | 2.3 | 3.3 |
| Optimize | 4 | 3.9 | 0.9 | 2.4 |
| NLS0017 (NRRL B-50931) | 5 | 1.8 | 1.4 | 1.6 |
| NLS0020 (NRRL B-50930) | 6 | 7.1 | 1.2 | 4.2 |
| NLS0064 (NRRL B-50938) | 7 | 3.4 | 1.9 | 2.7 |
| NLS0017/NLS0020 | 8 | 1.6 | 0.9 | 1.3 |
| NLS0038/NLS0020 | 9 | 1.3 | 1.2 | 1.3 |
| NLS0017 + Rhiz | 10 | 1.9 | 0.4 | 1.2 |
| NLS0020 + Rhiz | 11 | 3.9 | 1.4 | 2.7 |
| NLS0064 + Rhiz | 12 | 2.8 | 0.7 | 1.8 |
| Avg × Loc | | 4.0 | 1.4 | 2.7 |

### Example 2. Methylobacterium Application to Tomato

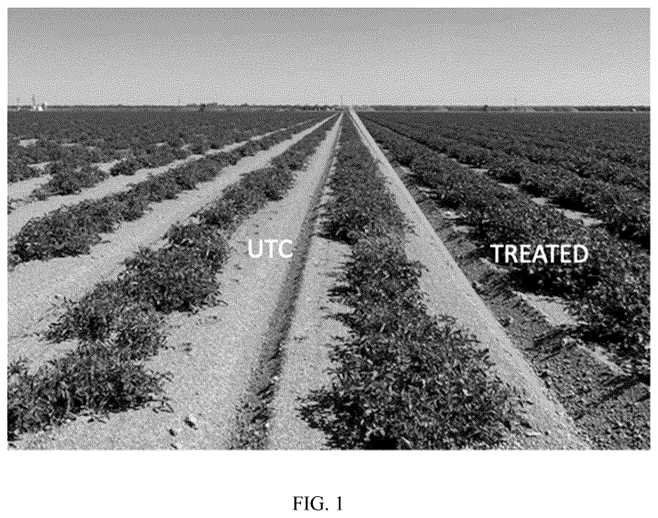

A tomato trial was established in California to evaluate effects of Methylobacterium strains applied as drench treatments prior to planting on tomato yield. A composition comprising NLS0017 and NLS0109 was applied to tomato seedlings as a drench prior to transplanting to a field for further growth and maturation. An infestation of beet curly top virus transmitted by beet leafhopper insects was observed to significantly decrease growth of untreated control plants, compared to the growth of plant treated with NLS0017 and NLS0109 prior to transplant to the field. Results are demonstrated in FIG. 1.

Leaf samples were collected from both Methylobacterium-treated and untreated portions of the field and stored at 4° C. until DNA extraction. Leaves were collected from one plant per row in a staggered manner to ensure a representative sample over the treatment area. A third-party lab used multiplex Polymerase Chain Reaction (PCR) to detect the presence of beet curly top virus (BCTV) and tomato spotted wilt virus (TSWV) in leaf tissue. The presence of BCTV was detected in 52.38% of non-treated control plants and only 18.18% of Methylobacterium inoculated plants, representing 34% fewer samples testing positive for the virus. The presence of TSWV was detected in 9.09% of non-treated control plants and 0.00% of Methylobacterium inoculated plants. Results are demonstrated in Table 4. Data were analyzed in JMP 16.2.0 using a nominal logistic regression with Chi-square post hoc test. This constitutes a statistically significant decrease in BCTV viral presence (p=0.017) and a non-significant decrease (p=0.0849) in TSWV viral presence associated with the application of Methylobacterium strains on tomato.

TABLE 4
Plant Sampling Results - BCTV and TSWV

| | | Detection of BCTV | | | | | | | |
|---|---|---|---|---|---|---|---|---|---|
| | | | Very | | | | Detection of TSWV | | |
| | | | Weak | Weak | Strong | Percent | | | Percent |
| | | Negative | Positive | Positive | Positive | Positive | Negative | Positive | Positive |
| Treatment | NLS0017 + | 18 | 2 | 0 | 2 | 18.18% | 22 | 0 | 0.00% |
| | NLS0109 | | | | | | | | |
| | (n = 22) | | | | | | | | |
| | NTC | 10 | 0 | 6 | 5 | 52.38%* | 19 | 2 | 9.09% |
| | (n = 21) | | | | | | | | |

### Example 3 Controlled Environment Analysis of Methylobacterium Treated Tomato Seedlings

Processing tomatoes are evaluated under controlled growth conditions to evaluate the ability of Methylobacterium NLS0017 and NLS0109 treatments comprising either strain alone or in compositions having varying ratios of each strain to provide resistance to viral disease by decreasing activity and/or viral presence of beet curly top virus (BCTV) vectored by beet leafhoppers. Tomato seeds will be planted in pairs and maintained under standard growth conditions. After germination, the weakest/smallest of each pair will be removed and the remaining seedlings grown for about 2 weeks to transplant size. Plants will then be randomly sampled for germination performance using baseline hyperspectral imaging to obtain a baseline element composition. Treatments will be applied to the plants after imaging as either a soil drench (“Drench”) or by addition to the top soil (“Top”) using a pipette. Both Drench and Top will include water only control treatments and four blends of Methylobacterium strains NLS0017 and NLS0109:
100-0 mixture of NLS0017 and NLS0109
75-25 mixture of NLS0017 and NLS0109
25-75 mixture of NLS0017 and NLS0109
0-100 mixture of NLS0017 and NLS0109
   A positive control for each of Drench and Top, will be a Bacillus subtilis treatment.

Plants will be subjected to hyperspectral imaging 7 and 14 days after treatments to assess average reflectance profiles in response to treatments. At each time point, individual plants will be sampled randomly and used to obtain composition data.

Resistance of tomato plants to BCTV infection will be assessed as follows. Tomato plants will be selected from trays used in germination performance. Bioassays will be performed with Viruliferous beet leafhoppers (from a specific colony). Viruliferous status of beet leafhoppers will be confirmed by loop mediated isothermal amplification (LAMP) analysis prior to testing.

For non-choice assays, viruliferous beet leafhoppers will be transferred to a clear plastic tube with one tomato leaflet in either end (both leaflets from the same treatment). Numbers of probing events (times beet leafhoppers inserted their mouthparts into vascular tissue) will be determined by staining. Bioassay's will be conducted at three time points, 7-14 days apart, to evaluate the effects of bacterial inoculant treatments. A total of 15 replications will be performed.

For choice bioassays, viruliferous beet leafhoppers will be used and transferred to a clear plastic tube with one tomato leaflet in either end, with one of the leaflets being an untreated control and the other being a leaflet from a plant treated with Methylobacterium or control inoculants. The numbers of probing events will be determined by staining. Bioassays will be conducted at three time points to evaluate the effects of bacterial inoculant treatments. A total of 15 replications will be performed.

The above experiments will identify Methylobacterium treatments and ratios of strains that provide the highest level of resistance to BCTV infection. BCTV resistance will be further evaluated using the best Methylobacterium treatment identified. Tomato plants will be placed inside meshed cages in a grid, and viruliferous beet leafhoppers will be released into each cage. Three tomato plant configurations will be examined:
All tomato plants will have had no Methylobacterium treatment
All tomato plants will have had “best” Methylobacterium treatment, as determined from non-choice and choice bioassays
About half of tomato plants will have had no Methylobacterium treatment and about half will have had “best” Methylobacterium treatment, as determined from non-choice and choice bioassays
   Approximately three weeks after infestation, leaf material from all plants will be subjected to LAMP analysis to determine BCTV infection. Six replicated cages will be used for all three treatments.

The breadth and scope of the present disclosure should not be limited by any of the above-described embodiments, but should be defined only in accordance with the following claims and their equivalents.

What is claimed:

1. A method for reducing insect damage and/or insect transmitted disease damage to a crop, wherein said insect is a piercing-sucking insect, and wherein said method comprises: (i) applying a composition comprising Methylobacterium strain NLS0017, deposited as NRRL B-50931 and/or NLS0109, deposited as NRRL B-67340 to a plant, a seed, or soil where seed has been or will be planted, and (ii) growing a crop in the presence of said piercing-sucking insect, thereby reducing damage to the plant from said insect and/or disease transmitted by said insect as compared to a plant not treated with said composition or grown in soil not treated with said composition.

2. The method of claim 1 wherein said insect damage is damage to plant tissue caused by insect feeding.

3. The method of claim 1 or 2 wherein said insect is a piercing-sucking insect vectors a pathogen that causes a plant disease.

4. The method of any one of claims 1 to 3 wherein said piercing-sucking insect is an aphid, a thrips or a leafhopper.

5. The method of claim 3 or 4 wherein said pathogen is a virus.

6. The method of claim 5 wherein said virus is a tospovirus or a geminivirus.

7. The method of any one of claims 1 to 6 wherein the composition comprises Methylobacterium strains NLS0017 and NLS0109.

8. The method of any one of claims 1 to 7 wherein the composition is applied to soil by broadcasting the composition, by drenching the soil with the composition, and/or by depositing the composition in a furrow where seed is or will be planted.

9. The method of any one of claims 1 to 7 wherein the composition is applied to a plant as a seed treatment, as a foliar spray, or as a pre-plant drench of a plant seedling.

10. The method of any of claims 7 to 9 wherein said composition comprises NLS0017 and NLS0109 as a 75:25 mixture of NLS0017 and NLS0109.

11. The method of any one of claims 7 to 9 wherein said composition comprises NLS0017 and NLS0109 as a 25:75 mixture of NLS0017 and NLS0109.

12. A method for reducing virus damage caused by a virus vectored by a leafhopper or thrips to a plant crop, wherein said method comprises: (i) applying a composition comprising Methylobacterium strain NLS0017, deposited as NRRL B-50931, and/or NLS0109, deposited as NRRL B-67340, to a plant, a seed, or soil where seed has been or will be planted, wherein said leafhopper or thrips is capable of vectoring a virus that infects said plant or a crop grown from said seed, and (ii) growing a crop of plants from said seed in the presence of said leafhopper or thrips, thereby reducing damage to the crop from said virus as compared to a crop not treated with said composition or grown in soil not treated with said composition.

13. The method of claim 12 wherein said crop is a tomato crop or peanut crop.

14. The method of claim 12 or 13 wherein the thrips is a western flower thrips or a tobacco thrips.

15. The method of any one of claims 12 to 14 wherein said virus is a tospovirus or a geminivirus.

16. The method of claim 15 wherein said tospovirus is tomato spotted wilt virus.

17. The method of claim 12 wherein said leafhopper is a beet leafhopper.

18. The method of claim 15 wherein said geminivirus is a beet curly top virus.

19. The method of any one of claims 12 to 18 wherein the composition is applied to soil by broadcasting the composition, by drenching the soil with the composition, and/or by depositing the composition in a furrow where said seed is or will be planted.

20. The method of claim 19 wherein the composition is applied to soil by depositing the composition in the furrow where the seed is or will be planted and deposited in the furrow prior to, concurrent with, or after placing seed in the furrow.

21. The method of any one of claims 12 to 18 wherein the composition is applied to said plant as a seed treatment, as a foliar spray, or as a pre-plant drench of a plant seedling.

22. The method of any one of claims 12 to 21 wherein the composition comprises NLS0017 and NLS0109.

23. The method of claim 22 wherein said composition comprises NLS0017 and NLS0109 as a 75:25 mixture of NLS0017 and NLS0109.

24. The method of claim 22 wherein said composition comprises NLS0017 and NLS0109 as a 25:75 mixture of NLS0017 and NLS0109.

25. A virus disease management method for use in growing a crop in a region where insects capable of transmitting a virus that infects said crop is present, wherein said method comprises: (i) applying a composition comprising Methylobacterium NLS0017 and/or NLS0109 to a plant, a seed, or soil where said seed has been or will be planted: (ii) applying an insecticide active against said insect to said plant, seed or soil where said seed has been or will be planted, and (iii) growing said plant or seed in the presence of said insect, thereby reducing damage to said plant from said virus as compared to a plant not treated with said composition in (i) or grown in soil not treated with said composition in (i).

26. The method of claim 25, wherein the amount of said insecticide is reduced in comparison to the recommended use for reducing insect populations in a crop grown in said region.

27. The method of claim 25 or 26 wherein said plant is a peanut plant.

28. The method of any one of claims 25 to 27 wherein said insect is a thrips.

29. The method of any one of claims 25 to 28 wherein said insect is a western flower thrips or a tobacco thrips.

30. The method of any one of claims 25 to 29 wherein said virus is a tospovirus.

31. The method of claim 30 wherein said tospovirus is tomato spotted wilt virus.

32. The method of any one of claims 25 to 31 wherein the composition is applied to soil by broadcasting the composition, by drenching the soil with the composition, and/or by depositing the composition in a furrow where peanut seed is or will be planted.

33. The method of claim 32 wherein the composition is applied to soil by depositing the composition in the furrow where the seed is or will be planted and deposited in the furrow prior to, concurrent with, or after placing peanut seed in the furrow.

34. The method of any one of claims 25, 26, and 30 to 33 wherein said plant is a tomato plant.

35. The method of claim 34 wherein said insect is a leafhopper.

36. The method of claim 35 wherein said insect is a beet leafhopper.

37. The method of any one of claims 364 to 36 wherein said virus is a geminivirus.

38. The method of claim 37 wherein said geminivirus is beet curly top virus.

39. The method of any one of claims 34 to 38 wherein the composition is applied to a tomato seedling as a drench.

40. The method of any one of claims 25 to 39 wherein said composition comprises NLS0017 and NLS0109.

41. The method of claim 40 wherein said composition comprises NLS0017 and NLS0109 as a 75:25 mixture of NLS0017 and NLS0109.

42. The method of claim 40 wherein said composition comprises NLS0017 and NLS0109 as a 25:75 mixture of NLS0017 and NLS0109.

43. The method of any one of claims 1 to 42 wherein said composition further comprises a strain of Rhizobium or Bradyrhizobium.
